(19) United States
(12) Patent Application Publication
(10) Pub. No.: US 2008/0305486 A1
(43) Pub. Date: Dec. 11, 2008

(54) SIGNAL AMPLIFICATION USING CIRCULAR HAIRPIN PROBES

(75) Inventors: WOEI TAN, Hercules, CA (US); Rick Moerschell, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 12/113,470

(22) Filed: May 1, 2008

Related U.S. Application Data
(60) Provisional application No. 60/942,312, filed on Jun. 6, 2007.

(51) Int. Cl. C12Q 1/68

(52) U.S. Cl. 435/6

(57) ABSTRACT

The present invention provides methods for detecting a target nucleic acid using a circular dual-hairpin probe that is formed upon the presence of the target nucleic acid. The detection methods find use in detecting the presence of antibody-antigen complexes and for detecting the binding of a ligand to its binding partner. Kits and reaction mixtures for performing the present methods are also provided.

### CROSS-REFERENCE TO RELATED APPLICATIONS

The present application claims the benefit of U.S. Provisional Application No. 60/942,312, filed on Jun. 6, 2007, the disclose of which is hereby incorporated herein by reference in its entirety.

### FIELD OF THE INVENTION

The present invention relates to improved detection of a target nucleic acid sequences using a circular polynucleotide.

### BACKGROUND OF THE INVENTION

A variety of methods have been used to enhance signal detection in immunoassays and detection of specific nucleic acid sequences (e.g., single polynucleotide polymorphisms). These methods commonly involve the use of fluorophore labels, enzyme conjugates and antibody-oligonucleotides conjugates. In most of these methods, signal enhancement is achieved by attaching multiple copies of fluorophore labels on an enzyme or an antibody conjugate, or by relying on downstream amplification of an oligonucleotide sequence attached to a target of interest (e.g., an antibody in a so-called immuno-polymerase chain reaction).

Previous oligonucleotide detection methods have relied on template-dependent ligation (see, e.g., U.S. Pat. Nos. 4,883,750; 4,988,617; 5,494,810; and 6,027,889). Also, oligonucleotide detection methods by others have required more than two probes, and in some approaches, both probes are required to hybridize to the template to complete formation of a circular DNA molecule (see, e.g., U.S. Pat. Nos. 4,883,750; 4,988,617; 5,494,810; and 6,027,889). Previously disclosed oligonucleotide detection methods are also limited by the use of longer probes of about 70-140 nucleotides in length (see, e.g., WO 99/049079).

There exists a need for improved methods to detect oligonucleotides, for example, in their use in detecting binding of ligand-binding partner binding pairs, in immunoassays or for the detection single nucleotide polymorphisms. The present invention addresses this and other needs.

### BRIEF SUMMARY OF THE INVENTION

In a first aspect, the invention provides methods for detecting a target nucleic acid in a sample. In some embodiments, the methods comprise:
a) contacting the sample with a hairpin extension polynucleotide under conditions such that if the target nucleic acid is present in the sample, the hairpin extension polynucleotide hybridizes to the target nucleic acid,
b) performing a template-dependent extension of the hairpin extension polynucleotide by at least two nucleotides to form a modified hairpin extension polynucleotide comprising a 3′-overhang of at least two nucleotides;
c) contacting the modified hairpin extension polynucleotide to a hairpin ligation polynucleotide in the presence of a ligase, wherein the hairpin ligation polynucleotide comprises a 3′-overhang of at least two nucleotides and the 3′-overhang of the hairpin ligation polynucleotide has the same number of nucleotides and is complementary to the 3′-overhang of the modified hairpin extension polynucleotide, such that the ligase ligates the 3′-end of the modified hairpin extension polynucleotide to the 5′-end of the hairpin ligation polynucleotide and ligates the 3′-end of the hairpin ligation polynucleotide to the 5′-end of the modified hairpin extension polynucleotide, wherein the ligation is a template independent ligation, thereby forming a circular polynucleotide; and
d) detecting the presence or absence of the circular polynucleotide, wherein the presence of the circular polynucleotide indicates the presence of the target nucleic acid in the sample.

In a further aspect, the invention provides methods of detecting an antigen in a sample. In some embodiments, the methods comprise:
a) contacting an antigen binding region of an antibody to the sample under conditions such that the antibody forms a complex with the antigen, if present, wherein the antibody is linked to a target oligonucleotide;
b) separating unbound antibody from the complex of the antibody and the antigen; and
c) detecting the complex of the antibody and the antigen, wherein the detecting step comprises:
   i) contacting the target oligonucleotide with a hairpin extension polynucleotide under conditions such the hairpin extension polynucleotide hybridizes to the target oligonucleotide,
   ii) performing a template-dependent extension of the hairpin extension polynucleotide by at least one nucleotide to form a modified hairpin extension polynucleotide comprising a 3′-overhang of at least one nucleotide;
   iii) contacting the modified hairpin extension polynucleotide to a hairpin ligation polynucleotide in the presence of a ligase, wherein the hairpin ligation polynucleotide comprises a 3′-overhang of at least one nucleotide and the 3′-overhang of the hairpin ligation polynucleotide has the same number of nucleotides and is complementary to the 3′-overhang of the modified hairpin extension polynucleotide, such that the ligase ligates the 3′-end of the modified hairpin extension polynucleotide to the 5′-end of the hairpin ligation polynucleotide and ligates the 3′-end of the hairpin ligation polynucleotide to the 5′ end of the modified hairpin extension polynucleotide, wherein the ligation is a template independent ligation, thereby forming a circular polynucleotide; and
   iv) detecting the presence or absence of the circular polynucleotide, wherein the presence of the circular polynucleotide indicates the presence of the complex of the antibody and the antigen

With respect to embodiments of the methods, in some embodiments, the circular polynucleotide is detected by contacting the circular polynucleotide with a primer and measuring a product of template-dependent extension of the primer.

In some embodiments, the template-dependent extension comprises the polymerase chain reaction.

In some embodiments, the template-dependent extension comprises isothermal amplification.

In some embodiments, the template-dependent extension comprises rolling circle amplification.

In some embodiments, the target nucleic acid is linked to an antibody.

In some embodiments, the product is detected by hybridizing the product to a complementary polynucleotide linked to a detectable reagent.

In some embodiments, the detectable reagent is a bead.

In some embodiments, the method is performed in a multiplex format.

In a related aspect, the invention provides kits. In some embodiments, the kits comprise:
a) a detection antibody attached to a target oligonucleotide
b) a hairpin extension polynucleotide that specifically hybridizes to the target oligonucleotide, wherein upon hybridization of the hairpin extension polynucleotide to the target oligonucleotide, template-dependent extension of the hairpin extension polynucleotide by at least one nucleotide forms a modified hairpin extension polynucleotide comprising a 3′-overhang of at least one nucleotide; and
c) a hairpin ligation polynucleotide comprising a 3′-overhang that specifically hybridizes to the 3′-overhang of the modified hairpin extension polynucleotide, thereby forming a circular polynucleotide.

In another aspect, the invention provides reaction mixtures. In some embodiment, the reaction mixtures comprise:
a) an antibody attached to an oligonucleotide
b) a hairpin extension polynucleotide that specifically hybridizes to the target oligonucleotide, wherein upon hybridization of the hairpin extension polynucleotide to the target oligonucleotide, template-dependent extension of the hairpin extension polynucleotide by at least one nucleotide forms a modified hairpin extension polynucleotide comprising a 3′-overhang of at least one nucleotide; and
c) a hairpin ligation polynucleotide comprising a 3′-overhang that specifically hybridizes to the 3′-overhang of the modified hairpin extension polynucleotide after template dependent extension of at least one nucleotide.

With respect to the embodiments of the kit and reaction mixture compositions, in some embodiments, the compositions further comprise a primer that hybridizes to a unique nucleotide sequence in the circular polynucleotide.

In some embodiments, the primer is attached to a fluorophore.

In some embodiments, the compositions further comprise a detectable oligonucleotide that hybridizes to a nucleic acid sequence amplified from the unique nucleotide sequence in the circular polynucleotide. In some embodiments, the detectable oligonucleotide is attached to a fluorophore. In some embodiments, the detectable oligonucleotide is attached to a bead.

In some embodiments, the compositions further comprise deoxynucleotide triphosphates (dNTPs) and a polymerase. In some embodiments, the compositions further comprise dideoxynucleotide triphosphates (ddNTPs).

In some embodiments, the compositions further comprise a plurality of detection antibodies attached to target oligonucleotides, a plurality of hairpin extension polynucleotides and a plurality of hairpin ligation polynucleotides sufficient for concurrently detecting a plurality of target oligonucleotides.

In some embodiments, each oligonucleotide attached to one of the plurality of antibodies has a different nucleic acid sequence. In some embodiments, each oligonucleotide attached to one of the plurality of antibodies has the same nucleic acid sequence.

In some embodiments, the compositions further comprise a capture antibody, wherein the capture antibody specifically binds to the same antigen as the detection antibody.

In some embodiments, the capture antibody is bound to a solid substrate.

### DEFINITIONS

The term “hairpin extension polynucleotide” refers to an oligonucleotide that forms a hairpin. In some embodiments, the hairpin extension polynucleotide can be about 60, 55, 50, 45, 40 or 35 nucleotide bases in length. The hairpin is formed by the complementary intramolecular annealing of 5′- and 3′-sequence segments, for example, over a length of about 4-20 base pairs, for example, about 5, 10 or 15 base pairs. The 3′-sequence segment of the hairpin extension polynucleotide can anneal to the target nucleic acid. The hairpin extension polynucleotide is designed such that the 3′-sequence segment favors annealing to the target nucleic acid over hairpin formation. For example, the annealing 3′-sequence segment can be about 4-20 nucleotides, for example about 5, 10 or 15 nucleotides. The 3′-end of the hairpin extension polynucleotide is subject to extension after annealing to the target nucleic acid to form a 3′-overhang that can anneal with the 3′-overhang of a hairpin ligation polynucleotide. Where the location of a single nucleotide polymorphism (SNP) in the target nucleic acid is known, the hairpin extension polynucleotide anneals to a contiguous nucleic acid segment immediately 5′ to the SNP location (e.g., with 1, 2, 3, 4 or 5 nucleotide bases), such that successful extension of the 3′-end of the hairpin extension polynucleotide would anneal to the SNP base.

The term “modified hairpin extension polynucleotide” refers to a hairpin extension polynucleotide that has annealed or hybridized to a target nucleic acid and been subjected to a 3′-extension reaction. A modified hairpin extension polynucleotide has additional nucleotides added to the 3′-terminus in comparison to an unmodified hairpin extension polynucleotide. That is, a modified hairpin extension polynucleotide has a 3′-overhang. In some embodiments the 3′-overhang is at least one nucleotide base. In some embodiments the 3′-overhang is at least two nucleotide bases.

The term “hairpin ligation polynucleotide” refers to an oligonucleotide that forms a hairpin and has a 3′-overhang that can anneal to the 3′-overhang of a modified hairpin extension polynucleotide. In some embodiments the 3′-overhang is at least one nucleotide base. In some embodiments the 3′-overhang is at least two nucleotide bases. In some embodiments, the hairpin ligation polynucleotide can be about 60, 55, 50, 45, 40 or 35 nucleotide bases in length. The hairpin is formed by the complementary intramolecular annealing of the 5′- and 3′-sequence segments, over a length of about 4-20 base pairs, for example about 5, 10 or 15 base pairs. The hairpin ligation polynucleotide typically does not anneal to the target nucleic acid. The 3′-overhang of the hairpin ligation polynucleotide and the 3′-overhang of the modified hairpin extension polynucleotide undergo intermolecular template-independent ligation when complementary.

The term “circular polynucleotide” refers to the oligonucleotide formed when the 3′-overhang of the hairpin ligation polynucleotide and the 3′-overhang of the modified hairpin extension polynucleotide intermolecularly anneal and the two polynucleotides are ligated to form a polynucleotide without a free 5′- or 3′-end.

The phrase “conditions for hybridization” refers to reaction conditions sufficient to allow a hairpin extension polynucleotide to anneal to a target nucleic acid. The conditions sufficient for hybridization will depend on temperature, salt, and the length and composition of the nucleic acid sequence segment to be annealed. Usually a temperature is selected that is about 5° C. less than the calculated melting temperature of the sequence segment to be hybridized. The melting temperature of a nucleic acid sequence segment can be readily determined using available algorithms (e.g., those available through Integrated DNA Technologies on the worldwide web at idtdna.com). Conditions sufficient for hybridization are generally known in the art and are described in basic laboratory treatises, for example, Sambrook and Russell, Molecular Cloning: A Laboratory Manual, 3^{rd} Edition, 2001, Cold Spring Harbor Press and Ausubel, et al., Current Protocols in Molecular Biology, 1987-2007, John Wiley & Sons.

The term “template-independent ligation” refers to intermolecular ligation of the hairpin extension polynucleotide and the hairpin ligation polynucleotide that occurs without the hairpin ligation polynucleotide annealing to the target nucleic acid.

The terms “oligonucleotide” or “polynucleotide” or “nucleic acid” interchangeably refer to a polymer of monomers that can be corresponded to a ribose nucleic acid (RNA) or deoxyribose nucleic acid (DNA) polymer, or analog thereof. This includes polymers of nucleotides such as RNA and DNA, as well as modified forms thereof, peptide nucleic acids (PNAs), locked nucleic acids (LNA™), and the like. In certain applications, the nucleic acid can be a polymer that includes multiple monomer types, e.g., both RNA and DNA subunits.

A nucleic acid is typically single-stranded or double-stranded and will generally contain phosphodiester bonds, although in some cases, as outlined herein, nucleic acid analogs are included that may have alternate backbones, including, for example and without limitation, phosphoramide (Beaucage et al. (1993) Tetrahedron 49(10):1925 and the references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26:1419), phosphorothioate (Mag et al. (1991) Nucleic Acids Res. 19:1437 and U.S. Pat. No. 5,644,048), phosphorodithioate (Briu et al. (1989) J. Am. Chem. Soc. 111:2321), O-methylphophoroamidite linkages (Eckstein, Oligonucleotides and Analogues: A Practical Approach, Oxford University Press (1992)), and peptide nucleic acid backbones and linkages (Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207), which references are each incorporated by reference. Other analog nucleic acids include those with positively charged backbones (Denpcy et al. (1995) Proc. Natl. Acad. Sci. USA 92:6097); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labeling moieties, or to alter the stability and half-life of such molecules in physiological environments.

In addition to naturally occurring heterocyclic bases that are typically found in nucleic acids (e.g., adenine, guanine, thymine, cytosine, and uracil), nucleic acid analogs also include those having non-naturally occurring heterocyclic or other modified bases, many of which are described, or otherwise referred to, herein. In particular, many non-naturally occurring bases are described further in, e.g., Seela et al. (1991) Helv. Chim. Acta 74:1790, Grein et al. (1994) Bioorg. Med. Chem. Lett. 4:971-976, and Seela et al. (1999) Helv. Chim. Acta 82:1640, which are each incorporated by reference. To further illustrate, certain bases used in nucleotides that act as melting temperature (Tm) modifiers are optionally included. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela, which is incorporated by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found, for example, in Tijssen, Techniques in Biochemistry and Molecular Biology—Hybridization with Nucleic Probes, “Overview ofprinciples of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

The term “ligand” as used herein refers to a polypeptide molecule that binds specifically to an analyte. Ligand includes antibodies, and non-antibody specific binding agents or “antibody mimics” that use non-immunoglobulin protein scaffolds as alternative protein frameworks for the variable regions of antibodies. Specific binding ligands with non-immunoglobulin scaffolds include those based on cytochrome b562 (Ku et al., Proc. Natl. Acad. Sci. U.S.A. 92(14):6552-6556 (1995)), fibronectin (U.S. Pat. Nos. 6,818,418 and 7,115,396), lipocalin (Beste et al. (Proc. Natl. Acad. Sci. U.S.A. 96(5):1898-1903 (1999)), calixarene (U.S. Pat. No. 5,770,380), A-domains (e.g., U.S. Patent Publication Nos. 2004/0175756, 2005/0048512, 2005/0053973, 2005/0089932 and 2005/0221384). Additional non-immunoglobulin ligands include those described, for example, in U.S. Pat. No. 5,260,203, Murali et al. (Cell. Mol. Biol. 49(2):209-216 (2003)).

An “antibody” refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively. As used in this application, an “antibody” encompasses all variations of antibody and fragments thereof that possess a particular binding specifically, e.g., for DR5. Thus, within the scope of this concept are full length antibodies, chimeric antibodies, single chain antibodies (ScFv), Fab, Fab′, and multimeric versions of these fragments (e.g., F(ab′)₂) with the same binding specificity.

The term “antigen” refers to a substance that when introduced into the body of an animal with an immune system stimulates the production of an antibody. An antigen can be a polypeptide, but may be a non-proteinaceous substance, for example, a nucleic acid, a carbohydrate, a small organic compound. An antibody specifically binds to an antigen.

The terms “bind(s) specifically” or “specifically bind(s)” interchangeably refer to the preferential association of an antibody, in whole or part, with a target antigen in comparison to non-target antigens. It is, of course, recognized that a certain degree of non-specific interaction may occur between an antibody and a non-target antigen. Nevertheless, specific binding, may be distinguished as mediated through specific recognition of the target antigen. Typically specific binding results in a much stronger association between the delivered molecule and an entity (e.g., an assay well or a cell) bearing the target antigen than between the bound antibody and an entity (e.g., an assay well or a cell) lacking the target antigen. Specific binding typically results in greater than about 10-fold and most preferably greater than 100-fold increase in amount of bound antibody (per unit time) to a cell or tissue bearing the target antigen as compared to a cell or tissue lacking the target antigen. Specific binding between two entities generally means an affinity of at least 10⁶ M⁻¹. Affinities greater than 10⁸ M⁻¹ are preferred. Specific binding can be determined using any assay for antibody binding known in the art, including Western Blot, ELISA, flow cytometry, immunohistochemistry.

The term “adaptor molecule” refers to a member of a high affinity binding pair. Exemplified adaptor molecule binding pairs include the high affinity interaction between biotin and an avidin (e.g., streptavidin, neutravidin, captavidin, etc, see, Molecular Probes Handbook on the worldwide web at invitrogen.com), staphylococcal proteins (e.g., protein A or protein G) and an immunoglobulin IgG constant region, an antibody and an antigen, a ligand (e.g., an antibody mimetic, e.g., A-domain, fibronectin binding domain (“Adnectin”) and other binding scaffolds known in the art and described herein) and its specific binding partner; a lectin and its specific binding partner.

### BRIEF DESCRIPTION OF THE DRAWINGS

FIG. 1 illustrates amplified signal of antigen detection using an oligonucleotide-coupled detection ligand (e.g. an antibody).

FIG. 2 illustrates amplified signal of antigen detection using an oligonucleotide-coupled adaptor molecule (e.g., streptavidin) bound to a detection ligand.

FIG. 3 illustrates amplified signal of antigen detection using an oligonucleotide-coupled ligand that specifically binds to an adaptor molecule (e.g., streptavidin or biotin).

FIG. 4 illustrates multiplex bead-based detection of single nucleotide polymorphisms (SNPs).

FIG. 5 illustrates the specificity of formation of the circular polynucleotide.

### DETAILED DESCRIPTION

### 1. Introduction

The present invention provides improved methods for detecting a target nucleic acid sequence. The methods find use where a target nucleic acid is used as a platform to amplify a signal, for example, for detecting a single nucleotide polymorphism (SNP) or for detecting the interaction between two members of a binding pair, e.g., an antibody-antigen interaction in an immunoassay, where the binding pair member or antibody is linked to a specific oligonucleotide.

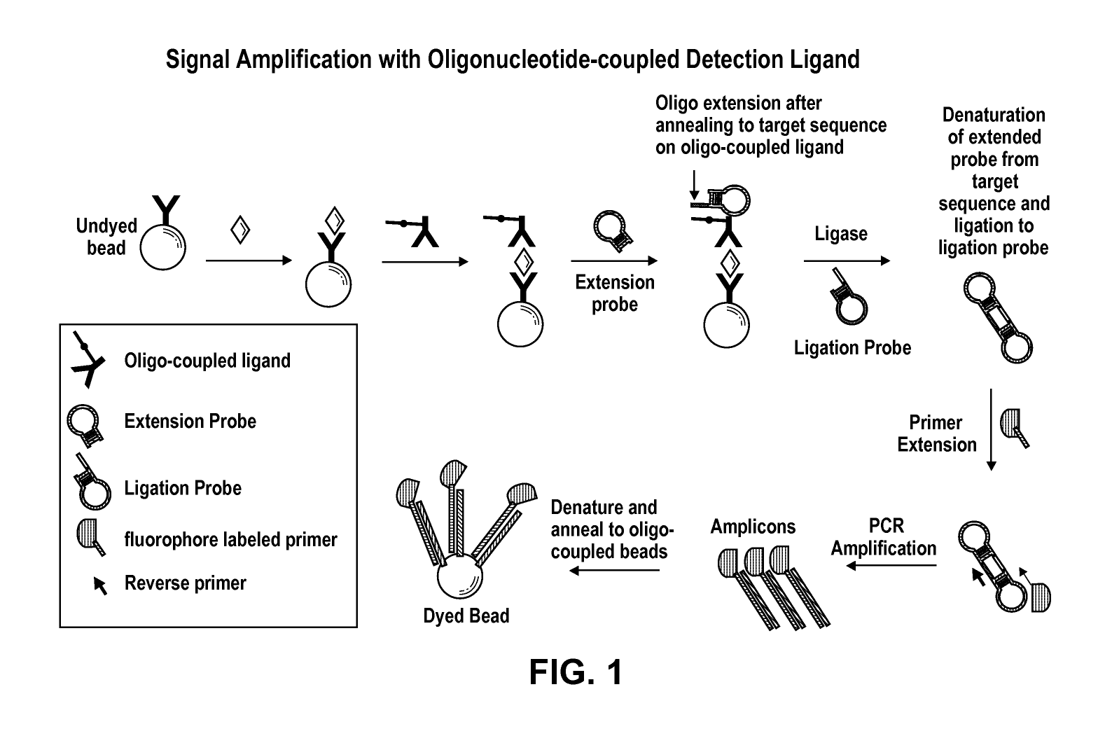

In the present detection methods, a target nucleic acid containing identifying (i. e., unique, signature) nucleotide bases is subject to detection. The target nucleic acid can be attached to an antibody or a member of a binding pair. To detect the target nucleic acid, a hairpin extension polynucleotide (i.e., extension probe, see FIG. 1) hybridizes to the target nucleic acid. The 3′-end of the hairpin extension polynucleotide is extended in a template-dependent manner to add nucleotide base pairs complementary to the target nucleic acid and including the identifying nucleotide bases, thereby forming a modified hairpin extension polynucleotide. The 3′-overhang of the hairpin extension polynucleotide is then annealed to the 3′-overhang of a hairpin ligation polynucleotide (i.e., ligation probe, see FIG. 1) and the two hairpin probes are ligated in a template independent manner to form a circular polynucleotide. Two probes are used overall, and only the hairpin extension polynucleotide hybridizes to the target nucleic acid sequence. A “zipcode” sequence segment unique to the circular polynucleotide is detected (e.g., by amplification) as evidence the formation of the circular polynucleotide and therefore, the underlying interaction between the two members of a binding pair, an antigen-antibody complex, or the presence of an SNP. The methods are well-suited for the concurrent detection and analysis of multiple samples.

### 2. Methods

a. Methods For Detecting Target Nucleic Acids

### i. Contacting a Sample with a Hairpin Extension Polynucleotide

The sample can be from any source that contains polynucleotides or target antigens. For example, the sample can be from an animal, a plant, bacterial, or fungal. The sample can be from a mammalian (e.g., human, primate, cat, dog) tissue or bodily fluid. The tissue sample can be non-invasive (e.g., from hair, inner cheek tissue) or can be from excised tissue, for example, from a biopsy. The bodily fluid can be from, for example but not limited to, blood, serum, sweat, tears, urine, saliva, etc. The sample can be a reaction mixture containing polynucleotides (e.g., a reaction mixture from an amplification reaction).

A tissue sample is processed according to methods well known in the art such that the polynucleotides are subject to detection. Kits for processing tissue samples (animal or plant) are commercially available, for example, from Qiagen, Valencia, Calif.

A sample may or may not contain a target nucleic acid or target antigen. A sample to be tested is suspected of having a target nucleic acid or target antigen. A positive control sample is known to contain a target nucleic acid or target antigen. A negative control sample is known not to contain a target nucleic acid or target antigen.

The target nucleic acid can be a known sequence or an unknown sequence. It can be synthetic or naturally obtained. If naturally obtained, the target nucleic acid sequence can be cut into convenient lengths, for example, using restriction enzymes. The target nucleic acid sequence will contain will contain one, two, or three contiguous nucleotides that are used to determine the presence or absence of the target nucleic acid. For example, a target nucleic acid sequence can have a single nucleotide polymorphism (SNP) or a single identifying nucleotide within its sequence that is detected using the present methods.

The target polynucleotide can be any length. In some embodiments, the target polynucleotide is less than about 100 nucleotide bases, for example, about 75, 50, 25 or 10 nucleotide bases, for example about 5-60, 30-50 or 35-45 nucleotide bases in length. In other embodiments, for example, when using genomic DNA samples, the target polynucleotide is longer than 100 nucleotide base pairs, for example, about 200, 500, 1000 nucleotide bases in length.

In some embodiments, for example, for ligand binding or immunoassays, the target nucleic acid is attached to a ligand molecule, either directly coupled or through one or more adaptor molecules. The ligand molecule can be an antibody mimetic or an immunoglobulin. Antibody mimetics, which bind a target molecule with an affinity comparable to an antibody, are known in the art, and include for example, single-chain binding molecules (U.S. Pat. No. 5,260,203), cytochrome b₅₆₂-based binding molecules (Ku et al (Proc. Natl. Acad. Sci. U.S.A. 92(14):6552-6556 (1995)), fibronectin or fibronectin-like protein scaffolds (“Adnectins,” see, U.S. Pat. Nos. 6,818,418 and 7,115,396), lipocalin scaffolds (Anticalin®, see, Beste et al. (Proc. Natl. Acad. Sci. U.S.A. 96(5):1898-1903 (1999)), calixarene scaffolds (U.S. Pat. No. 5,770,380), and A-domains and other scaffolds (see, U.S. Patent Publication No. 2006/0234299). In some embodiments, the ligand is an immunoglobulin. The immunoglobulin contains the variable region binding domains and can be, for example, a full-size antibody with constant regions, a FAb molecule, a single chain variable region, etc.

In the present methods, the sample is contacted with a hairpin extension polynucleotide under conditions sufficient for the hairpin extension polynucleotide to specifically hybridize to a target nucleic acid in the sample. In some embodiments, the conditions are sufficient for stringent hybridization. Conditions for stringent hybridization are known in the art, and are described, for example, in Sambrook and Russell, Molecular Cloning: A Laboratory Manual, 3^{rd} Edition, 2001, Cold Spring Harbor Laboratory Press; Ausubel, Current Protocols in Molecular Biology, 1987-2007, John Wiley Interscience, and herein.

The hairpin extension polynucleotide is designed to anneal to the target nucleic acid immediately 5′ to the identifying nucleotide bases in the target nucleic acid, so that when the 3′-end of the hairpin extension polynucleotide is extended, the added nucleotide bases are complementary to the identifying nucleotide bases. The hairpin stem of the extension probe opens to hybridize to the target nucleic acid on the oligo-coupled ligand. Generally, the Tm of the intramolecular hybridization of the hairpin stem of the hairpin extension polynucleotide will be lower than the Tm of the intermolecular hybridization of the 3′ sequence segment of the hairpin extension polynucleotide to the target nucleic acid.

### ii. Performing Template-Dependent Extension to Form a Modified Hairpin Extension Polynucleotide

Upon annealing to the target nucleic acid, the 3′-end of the hairpin extension polynucleotide is extended, e.g., at least one or at least two nucleotide bases. In some cases, the extension reaction is forced to terminate. In some cases, the extension reaction is forced to terminate by addition of a dideoxy-nucleotide (“ddNTP”) to the extension reaction mixture. The extension reaction is carried out according to methods well known in the art (see, e.g., Sambrook and Ausubel, supra). The extension reaction is performed under conditions sufficient to extend the 3′-end of the hairpin extension polynucleotide by the desired number of bases, e.g., at least one; at least two, etc. For example, a polymerase, a ddNTP and optionally one or more deoxynucleotides (dNTPs) can be added to the hybridization reaction mixture, above, creating an extension reaction mixture, and the extension reaction mixture is subject to a temperature that allows the polymerase for a time sufficient to extend the 3′-end of the hairpin extension polynucleotide by one or more nucleotide bases to yield a modified hairpin extension polynucleotide.

The temperature selected is dependent on the polymerase. In some embodiments, the extension temperature range is about 60-75° C., for example, about 65-72° C., for example about 60° C., 61° C., 62° C., 63° C., 64° C., 65° C., 66° C., 67° C., 68° C., 69° C., 70° C., 71° C., 72° C., 73° C., 74° C. or 75° C. Depending on the polymerase used, an extension reaction can also be carried out at room temperature, for example at about 25-37° C. The extension temperature can be held constant throughout the extension reaction or can be varied, as needed. An extension reaction extending the 3′-end of the hairpin extension polynucleotide can be completed in less than about 2 hours, for example, about 0.25, 0.5, 0.75, 1.0, 1.25, 1.5 hours. Any DNA polymerase can be used in the extension reactions, including for example, a DNA polymerase I, a Klenow fragment of a DNA polymerase I, a Taq polymerase, a T4 polymerase, a phi29 DNA polymerase, a VentR® DNA polymerase, and others known in the art.

In some embodiments, the 3′-terminus of the hairpin extension polynucleotide is extended by two or more nucleotides, for example, 2, 3, 4 or more nucleotide bases. In some embodiments, for example, when carrying out an assay to determine the binding of a ligand-binding partner binding pair or an immunoassay, the 3′-terminus of the hairpin extension polynucleotide is extended by one or more nucleotides, for example, 1, 2, 3, 4 or more nucleotide bases.

The extension reaction is template dependent; the nucleotide bases added are complementary to the target nucleic acid. The overhang created by the extended nucleotides will include the complementary one or more bases to the identifying one or more nucleotide bases in the target nucleic acid.

### iii. Ligating the Modified Hairpin Extension Polynucleotide to a Hairpin Ligation Polynucleotide to Form a Circular Polynucleotide

Following extension of the 3′-terminus of the hairpin extension polynucleotide (i.e., extension probe) to form a modified hairpin extension polynucleotide (i.e., modified extension probe), the modified hairpin extension polynucleotide released from the oligo-coupled ligand by thermal denaturation. The oligo-coupled ligand is removed by standard techniques, for example, phenol extraction. The remaining modified extension probe is hybridized then ligated to the hairpin ligation polynucleotide (i.e., ligation probe) to yield a circular polynucleotide.

The ligation reaction of the modified hairpin extension polynucleotide to the hairpin ligation polynucleotide is template independent. This is because the ligation reaction does not require that either the modified hairpin extension polynucleotide or the hairpin ligation polynucleotide be hybridized to the target nucleic acid at the time of ligation. Typically, the ligation hairpin polynucleotide does not hybridize to the target nucleic acid. Typically, only the hairpin extension polynucleotide anneals to the target nucleic acid, as discussed above.

Furthermore, the ligation of the modified hairpin extension polynucleotide to the hairpin ligation polynucleotide is stringent. That is, ligation between the 3′-overhang of the modified hairpin extension polynucleotide and the 3′-overhang of the hairpin ligation polynucleotide does not occur unless the overhangs are complementary. The complementary overhangs can be one, two, three or four nucleotide bases in length. The 3′-overhang of the hairpin ligation polynucleotide contains nucleotide bases that are identical to the identifying nucleotide bases in the target nucleic acid. Therefore, ligation depends on the extension of the 3′-end of the hairpin extension polynucleotide to produce an overhang that includes nucleotide bases that are complementary to the identifying nucleotide bases in the target nucleic acid.

The ligation reaction of the modified hairpin extension polynucleotide to the hairpin ligation polynucleotide is carried out under conditions sufficient to allow the modified hairpin extension polynucleotide to be ligated to the hairpin ligation polynucleotide. Such conditions are well known in the art (see, e.g., Sambrook and Ausubel, supra). Generally, ligation reactions performed at lower temperatures are carried out for longer periods of time. For example, a ligation reaction can be carried out at 4° C. overnight, at about 16° C. for 4-8 hours, or at room temperature (about 20-25° C.) for less than an hour, for example, about 10, 20, 30, 40 or 50 minutes. Ligase enzymes and ligase reaction buffers are commercially available, for example, from New England Biolabs, Ipswitch, Mass. or Promega, Madison, Wis. Ligase reaction mixtures will contain ATP.

### iv. Detecting the Presence or Absence of the Circular Polynucleotide

Formation of the circular polynucleotide can be detected using any method known in the art. For example, the circular polynucleotide can be detected by gel electrophoresis, restriction endonuclease digestion analysis, radioisotope detection (e.g., if ³²P-labelled or ³³P-labelled ATP is used in the ligase reaction). Other methods can also be employed.

In one embodiment, the circular polynucleotide contains a unique contiguous nucleotide sequence segment that can not be detected in unligated hairpin extension polynucleotide (modified or unmodified) or unligated hairpin ligation polynucleotide alone. The contiguous sequence segment unique to the circular polynucleotide is also referred to as a “zipcode” sequence. The zipcode sequence will encompass the nucleotide bases of the ligated 3′-overhangs. Therefore, a zipcode sequence will include the identifiable nucleotide nucleotide bases of the target nucleic acid sequence. Typically, the zipcode resides on the hairpin extension polynucleotide (i.e., extension probe) because it matches the specificity connoted by the target sequence. The hairpin ligation polynucleotide (i. e., ligation probe) can then be a universal probe, wherein four different probes are used; each with a different 5′ base. Exemplified embodiments of the methods are depicted in the Figures.

The zipcode sequence in a formed circular polynucleotide can be detected using any method known in the art. In some embodiments, the zipcode sequence is amplified. Amplification of a zipcode sequence can be performed using any techniques for nucleic acid amplification known in the art. Primers can anneal to a sequence segment on either the hairpin extension polynucleotide sequence segment or the hairpin ligation polynucleotide sequence segment and extend to include the zipcode sequence. Exemplified methodologies include polymerase chain reaction (PCR), isothermal amplification (ISA), rolling circle amplification (RCA), and in vitro transcription (IVT). See, for example, Ausubel, supra, PCR Primer: A Laboratory Manual, Dieffenbach, et al., eds, 2003, Cold Spring Harbor Laboratory Press; Nilsson, et al., Trends Biotechnol. (2006) 24(2):83-8; Zhang, et al., Clin Chim Acta. (2006) 363(1-2):61-70; Zhang, et al., Gene (1998) 211:277-85.

An amplified zipcode sequence (i.e., an amplicon) can be detected directly, for example, by amplifying from a labeled primer (e.g., a primer labeled with a radioisotope, a fluorophore, an enzyme, a chemiluminescent compound, etc.), or by incorporating labeled nucleotide bases into the amplified sequences. An amplified zipcode sequence can also be detected indirectly, for example, by hybridizing the amplified zipcode sequence to a labeled polynucleotide. The zipcode sequences can be about 4 to 20 bases long, for example, about 4, 5, 6, 7, 8, 9, 10, 11, 12, 13, 14 15, 16. 17, 18, 19 or 20 bases long. The labeled polynucleotide can be attached to, for example, a fluorophore, a radioisotope, an enzyme, a chemiluminescent compound or another detectable moiety. In some embodiments, the polynucleotide is attached to a fluorophore. Suitable fluorophores include the resorufin dyes, coumarin dyes, xanthene dyes, cyanine dyes, BODIPY dyes, pyrenes, and other fluorescent moieties. Exemplified fluorescent moieties for labeling a polynucleotide are commercially available, for example, from Invitrogen (Molecular Probes) and Amersham, and described in the Molecular Probes Handbook, available on the worldwide web at invitrogen.com. In one embodiment, the fluorophore is a rhodamine, for example, rhodamine green, rhodamine 6-G, rhodamine 101. In one embodiment, the fluorophore is Cy3, Alexa Fluor 532 or a phycoerythrin.

The detection resolution of a circular polynucleotide and a zipcode sequence within the circular polynucleotide can be increased by subjecting the ligation reaction mixture to an exonuclease enzyme. The exonuclease will digest any unligated hairpin extension polynucleotides and any unligated hairpin ligation polynucleotides.

In some embodiments, the directly or indirectly labeled amplified zipcode sequences are attached to a solid substrate, for example, a surface on an assay substrate (e.g., a multiwell plate, a chip) or a bead. One or multiple labeled polynucleotides can be attached to a solid substrate. In some embodiments, directly labeled amplified zipcode sequences are hybridized to complementary oligonucleotide sequences attached to a solid substrate (e.g., a multiwell plate, a chip, a bead).

In other embodiments, the amplified zipcode sequences are detected using real-time PCR, for example, using “molecular beacon” probes or similar detection methodologies, known in the art.

The labeled amplified zipcode sequences are then detected in a suitable instrument. Fluorescent labels can be detected, for example, using a luminometer, a fluorometer, a laser detection system, or a radioisotope detector. In some embodiments, the detection instruments are capable of simultaneously detecting multiple samples in a multi-well plate, for example 96-well, 192-well, 384-well, 768-well, 1536-well multi-well plates. For example, suitable luminometers and fluorometers are commercially available from, for example, Luminex, Austin Tex.; Thermo Fisher Scientific, Waltham, Mass.; and Turner Biosystems, Sunnyvale, Calif.

b. Methods For Detecting Antigens

The present methods are suitable for performing assays to evaluate the binding of receptor-ligand, ligand-binding partner binding pairs. The methods find use as a sensitive and efficient detection system for performing immunoassays. Accordingly, the present invention includes methods for detecting an antigen in a sample.

### i. Contacting an Oligonucleotide-Linked Antibody to a Sample

As discussed above, in ligand binding and immunoassays the target nucleic acid is attached to a ligand or an antibody, either directly or indirectly.

In one embodiment, the target nucleic acid is directly (e.g., covalently) coupled to the ligand or antibody. This can be accomplished using any method known in the art. For example, the target nucleic acid can be coupled to a ligand or an antibody using standard linkers, for example homo- and hetero-bifunctional linkers. Exemplified hetero-bifunctional linkers include succinimidyl 4-N-maleimidomethyl cyclohexane-1-carboxylate (SMCC) or Sulfosuccinimidyl 4-N-maleimidomethyl cyclohexane-1-carboxylate (Sulfo-SMCC)/N-Succinimidyl-S-acetylthioacetate (SATA) or N-Succinimidyl-S-acetylthiopropionate (SATP) or hydrazone/carbonyl. Bioconjugation moieties for use in linking oligonucleotides to ligands or antibodies are commercially available, for example, from Pierce Biotechnology, Rockford, Ill. In one embodiment, succinimidyl p-formylbenzoate (SFB) can be used to introduce benzaldehyde moieties to an amino-modified oligonucleotide. Succinimidyl 6-hydrazinonicotinic acetone hydrazone (SANH) can be used to introduce hydrazine moieties on the detection antibody. The hydrazine-modified detection antibody can then be reacted with a 5′-aldehyde modified oligonucleotide to form the oligo-coupled detection antibody. See, e.g., FIG. 1.

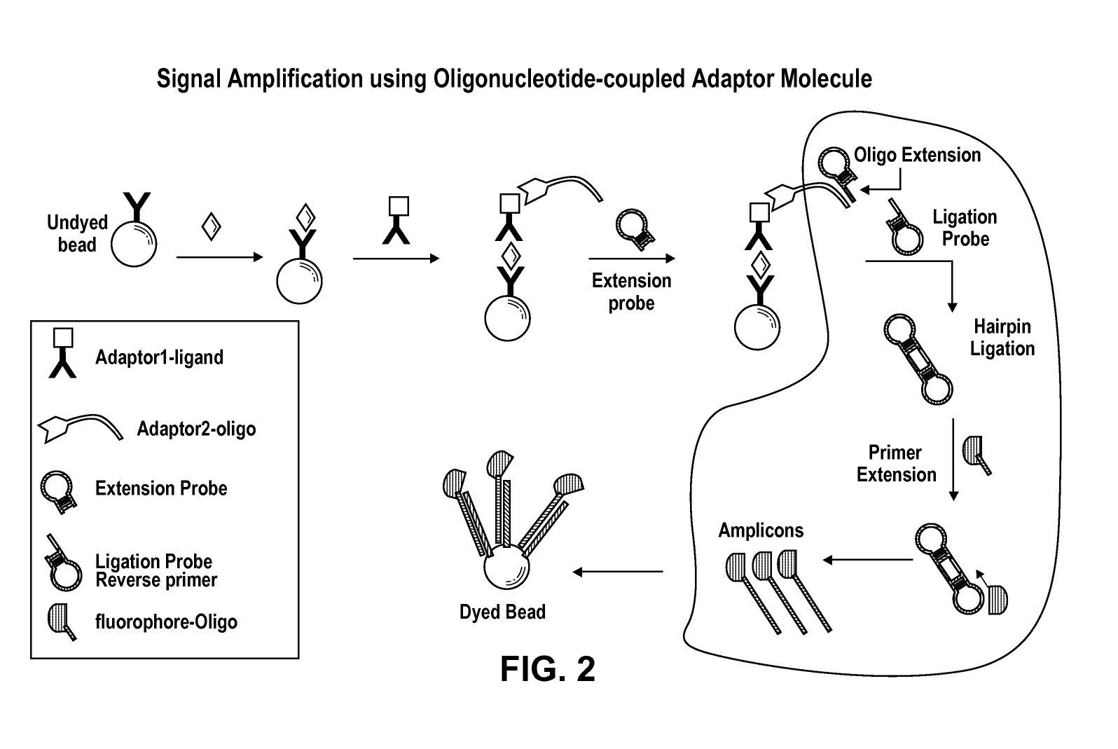

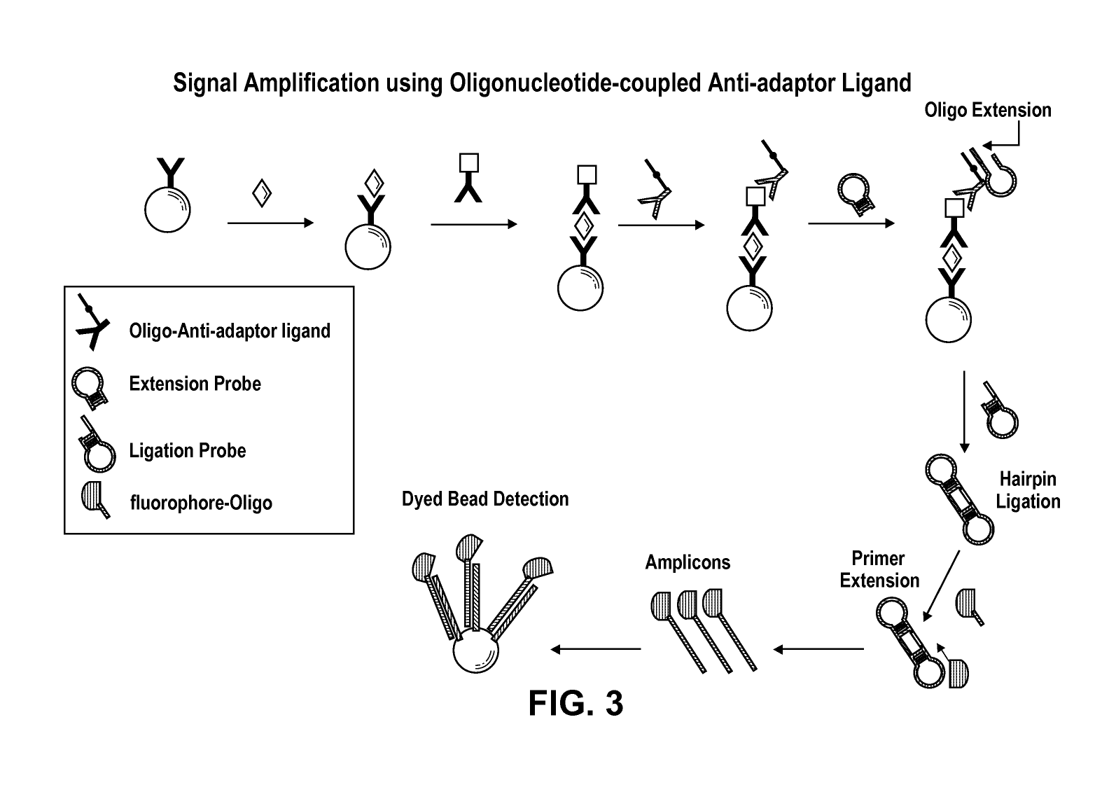

In other embodiments, the target nucleic acid is non-covalently coupled to the ligand or antibody. For example, the target nucleic acid can be coupled to a first member of an adaptor molecule binding pair (e.g., an avidin moiety, an antibody that specifically binds to the second member of the adaptor molecule binding pair) and the ligand or antibody can be coupled to a second member of an adaptor molecule binding pair (e.g., biotin). See, e.g., FIGS. 2 and 3.

The detection methods of the invention are compatible with any type of immunoassay or ligand-binding partner binding assay. For example, the immunoassays of the invention can be carried out in standard ELISA or sandwich capture format. In a standard ELISA format, the antigen of interest (e.g., in a sample) can first be coated on a substrate (e.g., a bead, a multiwell plate, an array chip), and then bound with a detection antibody, either directly or indirectly coupled to a target nucleic acid. In a sandwich capture format, the antigen of interest (e.g., in a sample) is first bound to a capture antibody, and then bound with a detection antibody, again either directly or indirectly coupled to a target nucleic acid. In some embodiments, the capture antibody is bound to a solid substrate, for example, a bead, a multiwell plate, an array chip, etc.). ELISA methodology is well known in the art. See, for example, The Elisa Guidebook, Crowther (Editor), Humana Press (2000). Protein array chips are available from, for example, Bio-Rad, Hercules, Calif.

In one embodiment, a capture ligand (e.g., antibody) is immobilized on a solid substrate (e.g., a bead, a multiwell plate, an array chip, etc.) and exposed to a sample containing a target agent (e.g., an antigen). The capture ligand binds to available target agent in the sample. Unbound material in the sample is washed away. The capture ligand-agent complex is then exposed to a binding partner of the agent (e.g., a detection antibody) that is directly coupled to a target oligonucleotide. The binding interaction of the capture ligand-agent-binding partner (e.g., capture antibody-antigen-detection antibody) ternary complex is detected through the presence of the target nucleic acid. See, FIG. 1.

In another embodiment, a capture ligand (e.g., antibody) is immobilized on a solid substrate (e.g., a bead, a multiwell plate, an array chip, etc.) and exposed to a sample containing a target agent (e.g., an antigen). The capture ligand binds to available target agent in the sample. Unbound material in the sample is washed away. The capture ligand-agent complex is then exposed to a binding partner of the agent (e.g., a detection antibody) that is directly coupled to a first binding partner of an adaptor molecule (e.g., an avidin moiety, a biotin moiety). The target oligonucleotide directly coupled to the second binding partner of the adaptor molecule (e.g., a biotin moiety, an avidin moiety, an antibody against the first binding partner of the adaptor molecule) is indirectly (i.e., non-covalently) bound to the detection binding partner of the agent through the adaptor molecule binding pair. The binding interaction of the capture ligand-agent-binding partner (e.g., capture antibody-antigen-detection antibody) ternary complex is detected through the presence of the target nucleic acid. See, FIGS. 2 and 3.

It will be recognized by those of skill in the art that generally between incubation steps for binding, unbound moieties (e.g., antigens, antibodies, ligands, binding partners) from a sample or reaction mixture are washed away with an appropriate buffer, e.g., phosphate-buffered saline or Tris-HCl comprising 1% or less of a non-ionic detergent, for example, Tween-80. Also, non-specific binding can be blocked or minimized, for example, with an unrelated protein, for example albumin.

Samples for detecting antigens of interest can be from any source suspected of containing the target antigen, as discussed above. The sample may be from a reaction mixture, or from a tissue or bodily fluid of a subject (e.g., an animal or a plant). In some embodiments, the sample is from a mammalian tissue or bodily fluid. For example, the sample can be from blood, serum, sweat, tears, saliva, urine or another bodily fluid. The mammal can be a human, a non-human primate, a domestic animal (e.g., canine or feline), an agricultural animal (e.g., equine, bovine, ovine, porcine), or a rodent (e.g., murine, rattus, lagomorpha, hamster, etc.). The tissue can be any corporeal tissue, for example from a biopsy. The sample may or may not contain the target antigen of interest.

### ii. Detecting Antibody-Antigen Complex

The binding of an antibody-antigen complex, or of a ligand specifically binding to its binding partner, is detected according to the steps outlined above. Detection of a ligand-agent or antibody-antigen complex typically will be carried out after unbound detection antibody or ligand binding partner has been washed away. The methods detect a target nucleic acid coupled to the detecting antibody or ligand. A hairpin extension polynucleotide is contacted with the target nucleic under conditions sufficient for hybridization. Upon hybridization, the 3′-terminus of the hairpin extension polynucleotide is extended by 1, 2, 3, 4, or more nucleotide bases in an extension reaction to yield a modified hairpin extension polynucleotide. The 3′-overhang of the modified hairpin extension polynucleotide is then hybridized to the 3′-overhang of a hairpin ligation polynucleotide. If the overhangs are complementary, then the modified hairpin extension polynucleotide and hairpin ligation polynucleotide can be ligated to form a circular polynucleotide. The ligation is template dependent because it proceeds without either the modified hairpin extension polynucleotide or the hairpin ligation polynucleotide being hybridized to the target nucleic acid. However, ligation is stringent and dependent on the extension of a 3′-overhang on the hairpin extension polynucleotide that is complementary to the 3′-overhang of a hairpin ligation polynucleotide.

The circular polynucleotide can be detected using any method known in the art, as discussed above. In one embodiment, a nucleic acid sequence segment unique to the formed circular polynucleotide (i.e., “a zipcode sequence”) is detected. The zipcode sequence can be detected by any method known in the art, including for example, amplification and hybridization technologies, described above. The methods for detecting antibody-antigen complexes or binding of a ligand to its binding partner can be performed in multiplex fully automated or partially automated systems, as described above.

c. Multiplex Methods

The methods are particularly suitable for the simultaneous detection of the presence or absence of multiple target nucleic acid molecules. As many as about 10, 100, 500, 1000, 1500, 2000 or more samples can be concurrently evaluated for one or more target nucleic acids using the present methods. Multiplex determinations can be conveniently carried out, for example, in commercially available multiwell plates, for example, 48-well, 96-well, 192-well, 384-well, 768-well, 1536-well multi-well plates, as discussed above. In other embodiments, multiplex determinations are carried out using an array chip.

Multiplex determinations can also be carried out under high-throughput conditions, in fully or partially automated systems. Automated systems that can be adapted for the present methods are available, for example, from Caliper Life Sciences, Hopkinton, Mass.

Multiplex determinations can be conveniently performed using a Bio-Plex® System (described on the worldwide web at bio-rad.com). Briefly, a Bio-Plex® System allows for the automated analysis of samples in 96-well multiwell plates (i.e., a microplate). Zipcode sequence amplicons amplified from a primer labeled with a fluorophore that anneals to the circular polynucleotide are hybridized to a detection oligonucleotide that is coupled to a detectable bead. In one embodiment, the beads in each of the 96 wells are internally labeled with two spectrally distinct fluorophores that emit a specific color and intensity uniquely indicative of the well location in the microplate (i.e., Luminex® xMAP® technology). The fluorophore labels on the bead and the zipcode amplicon are detected by flow cytometry. A fluidics system directs the beads from the microplates to be analyzed. The fluidics system aligns the beads from each well into single file for detection by a dual laser flow cytometry system. A first laser excites the fluorophores within the bead to identify the location in the microplate. A second laser excites the fluorophore attached to the amplified zipcode sequence. The detectors record and synthesize the information from the beads in each well, so that the signal from the amplified zipcode sequence is correlated with a particular location (i.e., sample, reaction mixture) in the microplate.

In the multiplex assay formats, the target nucleic acid sequences can be the same or different for each sample tested. It follows that the zipcode sequence created by formation of the circular polynucleotide also can be the same or different for each sample tested. For example, in one assay format, the target nucleic acid is the same and the assay for each sample tested is performed in four reaction mixtures, one for each nucleotide base (A, T, G, C). In this embodiment, the sequences of the hairpin extension polynucleotide and the hairpin ligation polynucleotide can be identical for each sample tested. A positive detection signal is detected in the assay reaction mixtures containing the appropriate dNTPs.

In another multiplex assay format, the target nucleic acid is known and attached to a detection antibody or ligand. A reaction mixture comprising one target nucleic acid sequence is exposed to a plurality of different samples, wherein each sample may or may not contain a target antigen. The reaction mixture comprises at least a target nucleic acid coupled to an antibody or ligand, and a hairpin extension polynucleotide. The hairpin ligation polynucleotide can be added to the reaction mixture with or without the presence of the target nucleic acid after carrying out the extension reaction. The target nucleic acid (i. e., the formed circular polynucleotide) is only detected in reaction mixtures where the detection antibody specifically binds to a target antigen. In some embodiments, a target antigen of interest is first isolated from a sample with a capture antibody, for example, in a “sandwich format” type immunoassay.

In a further multiplex assay format, one or more samples are exposed to two or more (i.e., a plurality) detection antibodies, wherein each detection antibody is attached to a different identifying target nucleic acid. The different target nucleic acids can be detected using the same or different hairpin extension polynucleotides, depending on the identifying (i.e., signature, unique) nucleotide bases within the target nucleic acids. Again, in some embodiments, the target antigens of interest can be first isolated from a sample with a capture antibody like in a “sandwich format” type immunoassay.

### 3. Kits

The invention also provides for kits comprising reagents for performing the present methods, particularly immunoassays. The kits comprise a detection ligand or binding partner (e.g., antibody) against a target agent (e.g., antigen) of interest, wherein the ligand (e.g., antibody) is coupled to a target oligonucleotide, directly or indirectly. The target oligonucleotide contains 1, 2, 3, 4 or more identifying nucleotide bases encompassed in a longer nucleic acid sequence segment, wherein the longer nucleic acid sequence segment will hybridize (i.e., is complementary to) the hairpin. The target oligonucleotide can be directly coupled to the detection antibody, as described above. In other embodiments, the target oligonucleotide is coupled directly to a first member of an adaptor molecule binding pair (e.g., an avidin) and the detection antibody is coupled directly to a second member of an adaptor molecule binding pair (e.g., biotin).

In other embodiments, for example, for SNP detection, the target oligonucleotide is uncoupled. For example, the target oligonucleotide can be in a sample or from a sample. In some embodiments, the target oligonucleotide is attached to an array chip, for example, a silica, glass, ceramic, metal, etc. chip. Such assay chips are known in the art and are commercially available, for example, from Affymetrix.

The kits can further comprise a hairpin extension polynucleotide that hybridizes to the target oligonucleotide 5′ to the identifying nucleotide bases and a hairpin ligation polynucleotide with a 3′-overhang that hybridizes and ligates to the extended 3′-overhang of a modified hairpin extension polynucleotide. The hairpin ligation polynucleotide typically does not hybridize to the target nucleic acid. In some embodiments, the hairpin extension polynucleotide and the hairpin ligation polynucleotide are less than about 60 nucleotide bases in length, for example, about 40-50 nucleotide bases in length.

The kits can further comprise a primer that specifically hybridizes to the circular polynucleotide formed by the ligation of a modified hairpin extension polynucleotide and a hairpin ligation polynucleotide. The primer specifically hybridizes to a sequence segment on the circular polynucleotide that is 5′ to the ligation junction. The primer may or may not be labeled with a detectably moiety (e.g., a radioisotope, a fluorophore, an enzyme, a chemiluminescent compound, etc.). Additionally, the kits can comprise a detectably labeled (e.g., with a radioisotope, a fluorophore, an enzyme, a chemiluminescent compound, a dyed bead, etc.) oligonucleotide that specifically hybridizes to an amplicon amplified from the primer. In some embodiments, the detectably labeled oligonucleotide is attached to a solid substrate, for example, a bead, a dyed bead, a multiwell plate. In some embodiments, the detectably labeled oligonucleotide is a molecular beacon.

The kits may also optionally comprise a capture ligand or binding partner (e.g., antibody) for binding the antigen of interest, for example, for a sandwich assay capture format. The capture ligand (e.g., antibody) can be immobilized on a solid substrate, for example, a bead, a multiwell plate, an array chip, etc. Some kits will also contain dNTPs, ddNTPs, appropriate buffers and co-factors (e.g., ATP), enzymes (e.g., polymerase, ligase), and instructions for use of the reagents to perform the methods. The kits can also contain one or more multiwell plates.

Kits that provide for carrying out multiplex assays can comprise a plurality (i.e., two or more) of different target nucleic oligonucleotides, each attached to a corresponding detection antibody or ligand. The different target nucleic oligonucleotides can be designed to differ only at the segment of identifying nucleotide bases, thereby allowing use of the same hairpin extension polynucleotide for each reaction mixture. However, in some kits, a plurality of different hairpin extension polynucleotides is included. The kits can also contain the same or a plurality of different hairpin ligation polynucleotides, depending on the number and nature of the target antigens to be detected.

### 4. Reaction Mixtures

The invention further provides reaction mixtures. The reaction mixtures include extension reaction mixtures, ligation reaction mixtures and detection reaction mixtures.

In some embodiments, the extension reaction mixtures comprise at least a target oligonucleotide coupled directly or indirectly (e.g., through an adaptor molecule) to a detection antibody that specifically binds to an antigen of interest, a hairpin extension polynucleotide that hybridizes to the target oligonucleotide immediately 5′ to the identifying nucleotide bases (described above), an extension polymerase, dNTPs and ddNTPs. Any DNA polymerase can be used in the extension reactions, including for example, a DNA polymerase I, a Klenow fragment of a DNA polymerase I, a Taq polymerase, a T4 polymerase, a phi29 DNA polymerase, a VentR® DNA polymerase, and others known in the art.

In other embodiments, for example, for SNP detection, the target oligonucleotide is uncoupled. For example, the target oligonucleotide can be in a sample or from a sample. In some embodiments, the target oligonucleotide is attached to an array chip, for example, a silica, glass, ceramic, metal, etc. chip. Such assay chips are known in the art and are commercially available, for example, from Affymetrix.

The ligation reaction mixtures comprise at least a modified hairpin extension polynucleotide with a 3′-overhang of 1, 2, 3 or 4 nucleotide bases, a hairpin ligation polynucleotide with a 3′-overhang that hybridizes and ligates to the 3′-overhang of a properly extended modified hairpin extension polynucleotide, a ligase and a buffer containing ATP. The target nucleic acid coupled to an antibody, directly or indirectly, may or may not be present.

The detection reaction mixtures (e.g., amplification reaction mixtures) comprise at least a circular polynucleotide formed by the ligation of a modified hairpin extension polynucleotide and a hairpin ligation polynucleotide, a primer that specifically anneals to the circular polynucleotide 5′ to the ligation junction, a polymerase and dNTPs. Appropriate DNA polymerases for use in the detection reaction mixtures are known in the art, including for example, a DNA polymerase I, a Taq polymerase, a T4 polymerase, a phi29 DNA polymerase, a VentR® DNA polymerase, and others known in the art. In some embodiments, the primer is labeled with a detectable marker (e.g., a radioisotope, a fluorophore, an enzyme, a chemiluminescent compound). In some embodiment, the detection reaction mixture further comprises a detectably labeled oligonucleotide that hybridizes to an amplicon amplified from the primer that specifically anneals to the circular polynucleotide. The detectably labeled oligonucleotide can be a molecular beacon. The detectably labeled oligonucleotide can be coupled to a fluorophore. In other embodiments, the detectably labeled oligonucleotide is attached to a solid substrate, for example a bead.

The reaction mixtures may also optionally comprise a capture ligand or binding partner (e.g., antibody) for binding the antigen of interest, for example, for a sandwich assay capture format. The capture ligand (e.g., antibody) can be immobilized on a solid substrate, for example, a bead or a multiwell plate. Some kits will also contain dNTPs, ddNTPs, appropriate buffers and co-factors (e.g., ATP), enzymes (e.g., polymerase, ligase), and instructions for use of the reagents to perform the methods. In some embodiments, the reaction mixtures are contained in one or more multiwell plates.

Reaction mixtures for carrying out multiplex assays can comprise a plurality (i.e., two or more) of different target nucleic oligonucleotides, each attached to a corresponding detection antibody or ligand. The different target nucleic oligonucleotides can be designed to differ only at the segment of identifying nucleotide bases, thereby allowing use of the same hairpin extension polynucleotide for each reaction mixture. However, in some reaction mixtures or reaction mixture replicates, a plurality of different hairpin extension polynucleotides is included. The reaction mixtures can also contain the same or a plurality of different hairpin ligation polynucleotides, depending on the number and nature of the target antigens to be detected.

### EXAMPLES

The following examples are offered to illustrate, but not to limit the claimed invention.

### Example 1

### Signal Amplification with Oligonucleotide-Coupled Detection Antibody

This example describes signal amplification from an olignucleotide-coupled antibody bound to antigen from ligated extension and ligation hairpin probes.

A target-specific oligonucleotide is covalently coupled to a detection antibody using, for example, a standard SMCC/SATA or hydrazone/carbonyl bioconjugation technique. For example, Succinimidyl p-formylbenzoate (SFB) is used to introduce benzaldehyde moieties to an amino-modified oligonucleotide. Succinimidyl 6-hydrazinonicotinic acetone hydrazone (SANH) is used to introduce hydrazine moieties on the detection antibody. The hydrazine-modified detection antibody is then reacted with a 5′-aldehyde modified oligonucleotide to form the oligo-coupled detection antibody. The oligonucleotide-coupled detection antibody is used to complete a sandwich, followed by a specific hybridization of a hairpin probe (“Extension Probe” or “hairpin extension polynucleotide”) to the oligonucleotide sequence. See, FIG. 1.

Using the oligonucleotide sequence as a template, one or more bases are extended on the hairpin. The extended bases can be in any combination of 3 bases, with the 4th base being a dideoxy-nucleotide. The extended hairpin (i.e., “modified hairpin extension polynucleotide”) anneals to a ligation probe (i.e., “hairpin ligation polynucleotide”) to form a circular molecule (i.e., “circular polynucleotide”). The specific nucleic acid sequence of the circular molecule serves as a template for subsequent signal amplification, for example, employing standard polymerase chain reaction, or other template amplification methods including isothermal amplification (IA), rolling circle amplification (RCA) and in vitro transcription (IVT). The formation of this circular template is highly specific given that the extension probe has to be extended correctly to allow the ligation probe to ligate (with ligase) and form the circular molecule.

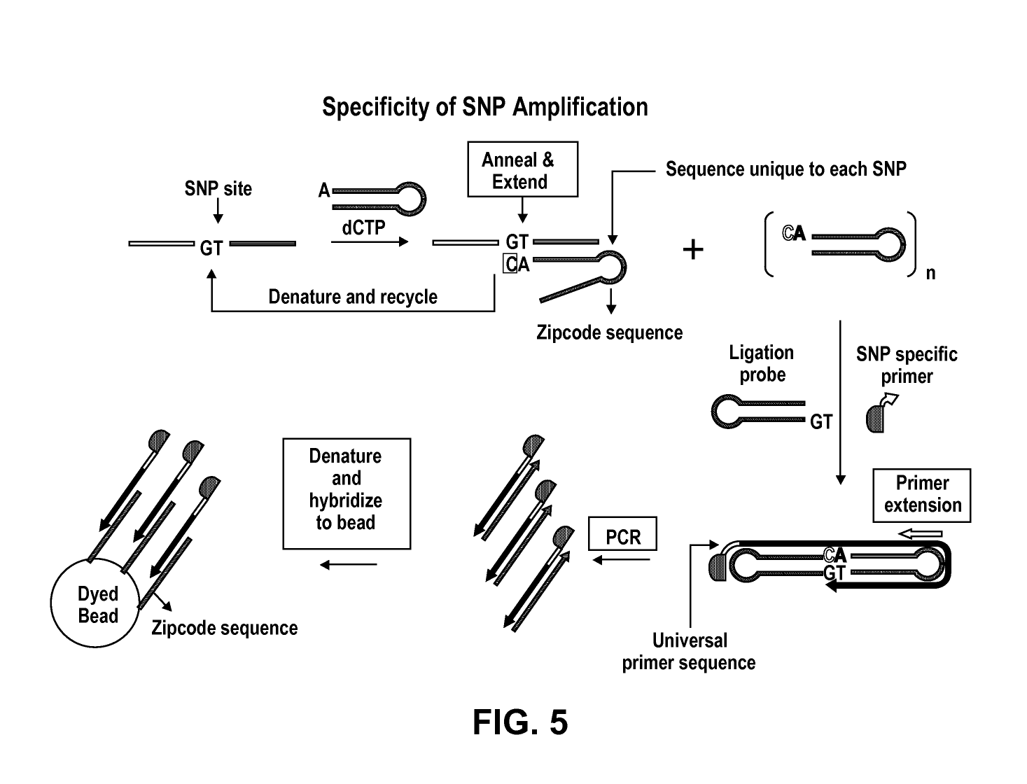

The formation of the circular molecule allows the amplification of target-specific amplicons. The amplicons generated from the amplification can be subjected to a multiplex bead based detection format (e.g., Bio-Plex). On a 96-well plate, each well accommodates simultaneous detection of multiple analytes. In the case of target antigens in an immunoassay, for each target detected, a circular product will be formed. A “zipcode sequence” located on the circular product permits specific amplicons to be amplified off the circular product and only the amplified product will hybridize to the its corresponding oligonucleotide-coupled bead. See, FIG. 5.

FIG. 2: Signal Amplification with Oligonucleotide-Coupled Streptavidin

This example describes signal amplification from an olignucleotide-coupled streptavidin bound to biotinylated antibody bound to antigen from ligated extension and ligation hairpin probes.

A format using adaptor molecules (e.g., avidin-biotin interactions) retains the current sandwich format using a biotinylated detection antibody. In this instance, a target specific thio-oligo-nucleotide is reduced by DTT treatment and followed by coupling to maleimide-derivatized streptavidin. This approach bypasses the coupling of the oligonucleotide to the detection antibody. Instead, a biotinylated antibody is bound to a streptavidin-oligonucleotide. The advantages of this approach include, (i) a more efficient coupling process and (ii) reduced chance of rendering the antibody inactive due to the harsh coupling procedure. See, FIG. 2.

### Example 3

### Signal Amplification with Oligonucleotide-Coupled Anti-Biotin Antibody

This example describes signal amplification from and olignucleotide-coupled anti-biotin antibody bound to a biotinylated antibody bound to an antigen from ligated extension and ligation hairpin probes.

This format employs an anti-biotin antibody for the oligonucleotide coupling process. In this case, the target specific oligonucleotide is coupled to an antibiotin antibody. This approach makes the oligonucleotide coupling process more universal and cost-effective, with the oligonucleotide being the only variable. See, FIG. 3.

### Example 4

### Multiplex Bead-Based Detection of Single Nucleotide Polymorphisms

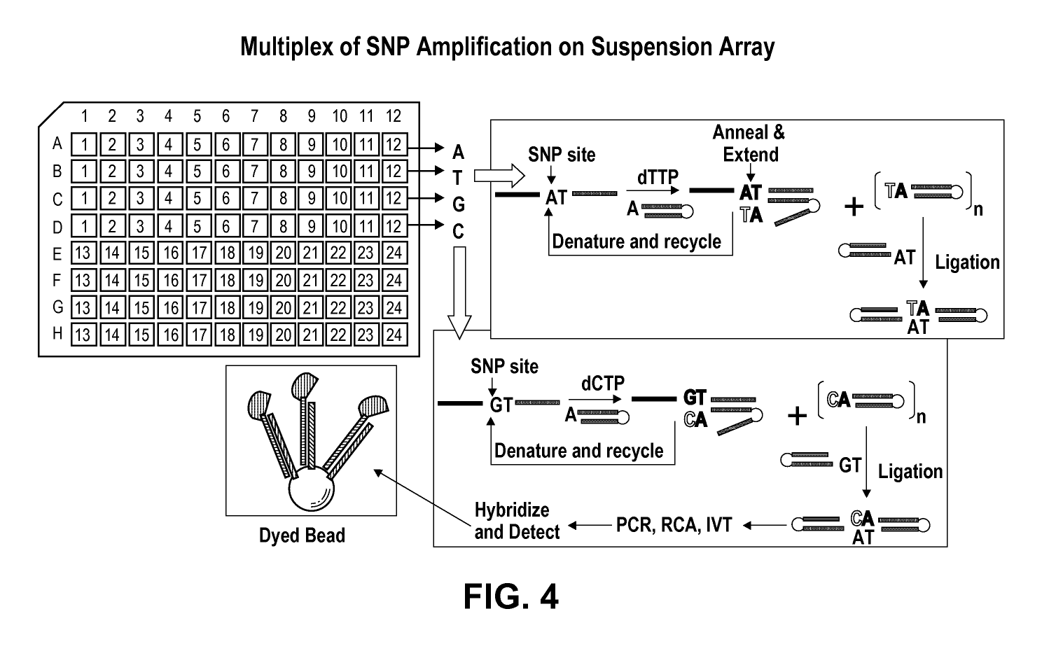

This format can be used to address SNP detection specifically. In this case, each quadruplex represents each sample tested for A, C, G and T extension. Assuming a 24-plex (24 SNPs) detection on 24 bead regions, each 96 well plate will accommodate 24 samples. Additional bead regions will be required to analyze more than 24 SNPs. Alternatively, the same sample can be split into additional wells if only 24 bead regions are used. To identify the type of SNP, each sample is split into four individual wells (A, B, C, D). To each well is added individual nucleotides (dATP, dGTP, dCTP or dTTP) for a probe extension reaction from a primer that anneals just 5′ of the potential SNP. Circular products formed following extension and ligation in each well identify the SNP (e.g., if the circular products formed in well 1A in FIG. 4, the identity of the SNP will be T). See, FIG. 4.

For each SNP detected, only one circular product is formed in one of the four wells. To confirm the formation of the circular products, only circular products are amplified. Only amplified sequence hybridize to the oligonucleotide-coupled beads. To further improve the specificity of this method, an exonuclease digestion step can be used to clean up the ligation preparation such that non-circular products are degraded.

To identify a SNP site, a zipcode sequence is incorporated into the detection probe specific for the SNP. Once the circular product is formed, the zipcode sequence can be amplified. The amplified zipcode sequence hybridizes to the sequence attached on the beads for fluorescent detection. To do multiplex SNP detections in each well, each SNP can have a detection probe with a unique zipcode sequence. Multiple detection probes can be added to each well. The number of detection probes added to each well is dependent on the number of bead regions available for multiplexing. For multiplexing, multiple circular products are formed and amplified simultaneously. To differentiate the amplified products, multiple beads are added. Each bead region is coupled with a zipcode sequence matching the detection probe where the SNP is located. The amplicons from the circular products will hybridize to the sequence on the beads for multiplex detection. Each bead region is specific to each SNP.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for detecting a target nucleic acid in a sample, the method comprising,
a) contacting the sample with a hairpin extension polynucleotide under conditions such that if the target nucleic acid is present in the sample, the hairpin extension polynucleotide hybridizes to the target nucleic acid,
b) performing a template-dependent extension of the hairpin extension polynucleotide by at least two nucleotides to form a modified hairpin extension polynucleotide comprising a 3′-overhang of at least two nucleotides;
c) contacting the modified hairpin extension polynucleotide to a hairpin ligation polynucleotide in the presence of a ligase, wherein the hairpin ligation polynucleotide comprises a 3′-overhang of at least two nucleotides and the 3′-overhang of the hairpin ligation polynucleotide has the same number of nucleotides and is complementary to the 3′-overhang of the modified hairpin extension polynucleotide, such that the ligase ligates the 3′-end of the modified hairpin extension polynucleotide to the 5′-end of the hairpin ligation polynucleotide and ligates the 3′-end of the hairpin ligation polynucleotide to the 5′-end of the modified hairpin extension polynucleotide, wherein the ligation is a template independent ligation, thereby forming a circular polynucleotide; and
d) detecting the presence or absence of the circular polynucleotide, wherein the presence of the circular polynucleotide indicates the presence of the target nucleic acid in the sample.

2. The method of claim 1, wherein the circular polynucleotide is detected by contacting the circular polynucleotide with a primer and measuring a product of template-dependent extension of the primer.

3. The method of claim 2, wherein the template-dependent extension comprises the polymerase chain reaction.

4. The method of claim 2, wherein the template-dependent extension comprises isothermal amplification.

5. The method of claim 2, wherein the template-dependent extension comprises rolling circle amplification.

6. The method of claim 1, wherein the target nucleic acid is linked to an antibody.

7. The method of claim 2, wherein the product is detected by hybridizing the product to a complementary polynucleotide linked to a detectable reagent.

8. The method of claim 7, wherein the detectable reagent is a bead.

9. The method of claim 1, wherein the method is performed in a multiplex format.

10. A method of detecting an antigen in a sample comprising:
a) contacting an antigen binding region of an antibody to the sample under conditions such that the antibody forms a complex with the antigen, if present, wherein the antibody is linked to a target oligonucleotide;
b) separating unbound antibody from the complex of the antibody and the antigen; and
c) detecting the complex of the antibody and the antigen, wherein the detecting step comprises:
i) contacting the target oligonucleotide with a hairpin extension polynucleotide under conditions such the hairpin extension polynucleotide hybridizes to the target oligonucleotide,
ii) performing a template-dependent extension of the hairpin extension polynucleotide by at least one nucleotide to form a modified hairpin extension polynucleotide comprising a 3′-overhang of at least one nucleotide;
iii) contacting the modified hairpin extension polynucleotide to a hairpin ligation polynucleotide in the presence of a ligase, wherein the hairpin ligation polynucleotide comprises a 3′-overhang of at least one nucleotide and the 3′-overhang of the hairpin ligation polynucleotide has the same number of nucleotides and is complementary to the 3′-overhang of the modified hairpin extension polynucleotide, such that the ligase ligates the 3′-end of the modified hairpin extension polynucleotide to the 5′-end of the hairpin ligation polynucleotide and ligates the 3′-end of the hairpin ligation polynucleotide to the 5′-end of the modified hairpin extension polynucleotide, wherein the ligation is a template independent ligation, thereby forming a circular polynucleotide; and
iv) detecting the presence or absence of the circular polynucleotide, wherein the presence of the circular polynucleotide indicates the presence of the complex of the antibody and the antigen

11. A kit comprising
a) a detection antibody attached to a target oligonucleotide
b) a hairpin extension polynucleotide that specifically hybridizes to the target oligonucleotide, wherein upon hybridization of the hairpin extension polynucleotide to the target oligonucleotide, template-dependent extension of the hairpin extension polynucleotide by at least one nucleotide forms a modified hairpin extension polynucleotide comprising a 3′-overhang of at least one nucleotide; and
c) a hairpin ligation polynucleotide comprising a 3′-overhang that specifically hybridizes to the 3′-overhang of the modified hairpin extension polynucleotide, thereby forming a circular polynucleotide.

12. The kit of claim 11, further comprising a primer that hybridizes to a unique nucleotide sequence in the circular polynucleotide.

13. The kit of claim 12, wherein the primer is attached to a fluorophore.

14. The kit of claim 11, further comprising a detectable oligonucleotide that hybridizes to a nucleic acid sequence amplified from the unique nucleotide sequence in the circular polynucleotide.

15. The kit of claim 14, wherein the detectable oligonucleotide is attached to a fluorophore.

16. The kit of claim 14, wherein the detectable oligonucleotide is attached to a bead.

17. The kit of claim 11, further comprising deoxynucleotide triphosphates (dNTPs) and a polymerase.

18. The kit of claim 17, further comprising dideoxynucleotide triphosphates (ddNTPs).

19. The kit of claim 11, further comprising a plurality of detection antibodies attached to target oligonucleotides, a plurality of hairpin extension polynucleotides and a plurality of hairpin ligation polynucleotides sufficient for concurrently detecting a plurality of target oligonucleotides.

20. The kit of claim 19, where each oligonucleotide attached to one of the plurality of antibodies has a different nucleic acid sequence.

21. The kit of claim 19, where each oligonucleotide attached to one of the plurality of antibodies has the same nucleic acid sequence.

22. The kit of claim 11, further comprising a capture antibody, wherein the capture antibody specifically binds to the same antigen as the detection antibody.

23. The kit of claim 22, wherein the capture antibody is bound to a solid substrate.

24. A reaction mixture comprising
a) an antibody attached to an oligonucleotide
b) a hairpin extension polynucleotide that specifically hybridizes to the target oligonucleotide, wherein upon hybridization of the hairpin extension polynucleotide to the target oligonucleotide, template-dependent extension of the hairpin extension polynucleotide by at least one nucleotide forms a modified hairpin extension polynucleotide comprising a 3′-overhang of at least one nucleotide; and
c) a hairpin ligation polynucleotide comprising a 3′-overhang that specifically hybridizes to the 3′-overhang of the modified hairpin extension polynucleotide after template dependent extension of at least one nucleotide.

25. The reaction mixture of claim 24, further comprising a primer that hybridizes to a unique nucleotide sequence in the circular polynucleotide.

26. The reaction mixture of claim 25, wherein the primer is attached to a fluorophore.

27. The reaction mixture of claim 24, further comprising a detectable oligonucleotide that hybridizes to a nucleic acid sequence amplified from the unique nucleotide sequence in the circular polynucleotide.

28. The reaction mixture of claim 27, wherein the detectable oligonucleotide is attached to a fluorophore.

29. The reaction mixture of claim 27, wherein the detectable oligonucleotide is attached to a bead.

30. The reaction mixture of claim 24, further comprising deoxynucleotide triphosphates (dNTPs) and a polymerase.

31. The reaction mixture of claim 30, further comprising dideoxynucleotide triphosphates (ddNTPs).

32. The reaction mixture of claim 24, further comprising a plurality of detection antibodies attached to target oligonucleotides, a plurality of hairpin extension polynucleotides and a plurality of hairpin ligation polynucleotides sufficient for concurrently detecting a plurality of target oligonucleotides.

33. The reaction mixture of claim 32, where each oligonucleotide attached to one of the plurality of antibodies has a different nucleic acid sequence.

34. The reaction mixture of claim 32, where each oligonucleotide attached to one of the plurality of antibodies has the same nucleic acid sequence.

35. The reaction mixture of claim 24, further comprising a capture antibody, wherein the capture antibody specifically binds to the same antigen as the detection antibody.

36. The reaction mixture of claim 35, wherein the capture antibody is bound to a solid substrate.
